(19) United States
(12) Patent Application Publication
(10) Pub. No.: US 2006/0161090 A1
(43) Pub. Date: Jul. 20, 2006

(54) Heel support apparatus and method of using

(76) Inventor: James C. Lee, Brea, CA (US)

Correspondence Address: MACPHERSON KWOK CHEN & HEID LLP, 1762 TECHNOLOGY DRIVE, SUITE 226, SAN JOSE, CA, 95110, US

(21) Appl. No.: 11/332,968

(22) Filed: Jan. 17, 2006

Related U.S. Application Data
(60) Provisional application No. 60/644,092, filed on Jan. 14, 2005.

(51) Int. Cl. A61F 13/00; A61F 13/06

(52) U.S. Cl. 602/66; 602/60; 602/61; 602/62

(57) ABSTRACT

In accordance with at least one embodiment of the present invention, a heel support apparatus includes a substantially flexible body member, a first fastening member, and a second fastening member. The body member has symmetry across a central first axis and includes both a first end and a second end and a first side and second side disposed oppositely on the first axis, where the first end has a first fastening site and a second fastening site, and a second end has a third fastening site and a fourth fastening site disposed symmetrically across the first axis. The first fastening member has a first end fixedly attached to the first fastening site and a second end selectively attached to the second fastening site. The second fastening member has a first end fixedly attached to the third fastening site and a second end selectively attached to the fourth fastening site.

### CROSS REFERENCE TO RELATED APPLICATION

The present invention relies for priority on a provisional patent application No. 60/644,092 filed in the United States Patent Office on Jan. 14, 2005, the entire content of which is hereby incorporated herein by reference.

### TECHNICAL FIELD

The present invention relates generally to medical devices, and more particularly to a heel support that provides medial and lateral compression to provide medical relief of heel and arch pain.

### RELATED ART

The incidence of heel pain in human patients is increasing at a fairly steady rate in the general population. Currently there are several professional treatments available for heel pain. Treatments can typically range from the application of a Low Dye strapping method to surgical release of the medial and central slips of the plantar fascia. These treatments may be inconvenient, painful, and/or expensive. The Low Dye method was first devised by Dr. Ralph Dye. This particular strapping method requires the intervention of a professional with an extensive knowledge in foot strapping methods and techniques. Once the Low Dye strapping is applied, the treatment is most effective for the first two days following application. The patient or other non-professional typically cannot make adjustments once the strapping is applied. The inability of effective self-strapping and the increased probability of professional visits make the Low Dye strapping undesirable for people suffering from heel pain due to plantarfasciitis, heel spurs, anterior tibial dysfunction, foot sprain, Achilles tendonitis and bursitis, arch pain, stress fractures of metatarsals, and avulsion fracture. The avulsion fracture is not to be confused with the Jones' fracture which is actually a perpendicular fracture within 1.5 cm of the fifth metarsarsal base. The Jones' fracture usually requires surgical and/or non-weight bearing short leg fiberglass cast. Further, the application of tape adhesives in the Low Dye strapping method may give rise to a potential allergic reaction in the patient.

The particular problem of pain due to plantarfasciitis is addressed by a Foot Alleviator, as described in U.S. Pat. No. 6,558,339, to Michael E. Graham. However, the Graham Foot Alleviator is inadequate for general application since, among other things, it fails to address the differing arch heights in individuals. Graham teaches the application of two pads on the underside of his foot alleviator, one pad positioned one under the arch and the other pad positioned under the outer aspect of the heel. This structure cannot accommodate the heel and arch of the general populace. Hence, Graham's solution has a limited applicability. The heel alleviator claims to provide relief of heel pain through structural re-positioning of the foot. However, unless the foot being treated with the heel alleviator meets strict criteria, the Graham apparatus will provide little or no relief.

Another attempt to provide relief from heel pain is through the use of a heel realigning sock (HRS). Further, the HRS may include a strap that attaches from an outside portion of the foot passing under the bottom of the heel and anchors to an inside portion of the foot. However, this structure will not provide lasting realignment of the heel bone. The HRS claims to provide realignment of the calcaneus portion of a foot that is not immobilized by a cast or other device. As a result, there remains a need in the art for convenient and effective treatments for heel pain.

### BRIEF DESCRIPTION OF THE DRAWINGS

FIG. 1 shows a perspective view of a heel support apparatus 102 applied to a human right foot 104, in accordance with an embodiment of the present invention.

FIG. 2 shows a side view of the heel support apparatus applied to a human left foot 202, in accordance with an embodiment of the present invention.

FIG. 3 shows a plan view of the heel support apparatus, in accordance with an embodiment of the present invention.

FIG. 4 shows an exemplary attachment of a first attachment member positioned to hold a first fastening site and a second fastening site on the body member at a first distance d1 to provide a first amount of tension across an operative portion of the first attachment member corresponding to a first amount of medial and lateral compression, in accordance with an embodiment of the present invention.

FIG. 5 shows an exemplary attachment of the first attachment member positioned to hold the first fastening site and the second fastening site on the body member at a second distance d2 to provide a second amount of tension across an operative portion of the first attachment member corresponding to a second amount of medial and lateral compression, in accordance with an embodiment of the present invention.

FIG. 6 shows an exemplary attachment of the first attachment member positioned to hold the first fastening site and the second fastening site on the body member at a third distance d3 to provide a third amount of tension across an operative portion of the first attachment member corresponding to a third amount of medial and lateral compression, in accordance with an embodiment of the present invention.

FIG. 7 shows a perspective view of the support in a closed position while not embracing a foot, in accordance with an embodiment of the present invention.

FIG. 8 shows a top view of the support in a closed position while not embracing a foot, in accordance with an embodiment of the present invention.

FIG. 9 shows a support apparatus application flow comprising a method of using the support, in accordance with an embodiment of the present invention.

Embodiments of the present invention and their advantages are best understood by referring to the detailed description that follows. It should be appreciated that like reference numerals are used to identify like elements illustrated in one or more of the figures. A more complete understanding of embodiments of the present invention will be afforded to those skilled in the art, as well as a realization of additional advantages thereof, by a consideration of the following detailed description.

### DETAILED DESCRIPTION

In accordance with one or more embodiments of the present invention, a heel support and method of using are disclosed, which may may begin to provide nearly instant relief from heel pain in a cost effective and convenient manner by applying medial and lateral compression of a user's heel, where the fat pad portion of a user's foot may be properly repositioned and the plantarfascia portion may be supported and relaxed. More particularly, the application of the disclosed apparatus and method may provide lasting relief from and prevent the recurrence of plantarfasciitis.

FIG. 1 shows a perspective view of a heel support apparatus 102, hereinafter support 102, applied to a human right foot 104, in accordance with an exemplary embodiment of the present invention. Support 102 includes a substantially flexible body member 106 that may be applied to embrace a lower portion 108 of foot 104 and to simultaneously apply medial and lateral compression of a heel portion 110 of foot 104 based on the cooperative actions of a first fastening member 112, a second fastening member 114, and flexible body member 106. Compression is applied due, in part, to the elastic properties of flexible body member 106 wherein portions of flexible body member 106 are stretched or deformed when applying and wearing support 102. The resiliency or tendency of flexible body member 106 to return to its original shape prior to deformation causes a contraction force on the fastening members (112, 114) and across the surface of flexible body member 106 that is operative in applying the medial and lateral compression.

In this disclosure, medial compression includes the application of a force 120 upon a rear portion 122 of foot 104, where force 120 is directed along an axis 124 oriented towards a forefoot portion 126 of foot 104. Medial compression is primarily applied by the cooperative actions of first fastening member 112 and flexible body member 106. Further, lateral compression includes the application of a pair of opposing forces (130, 132) along an axis 134 upon the side portions (136, 138) of the foot 104. Lateral compression is primarily applied by the cooperative actions of second fastening member 114 and flexible body member 106. Since support 102 may be applied to either a left foot or a right foot, the description of regions, sides, and portions are correspondingly applicable to either foot orientation. An individual may wear support 102 with or without shoes. Depending on the condition being treated, one or more embodiments of the present invention may be used in conjunction with a functional orthotic device or a walking cast.

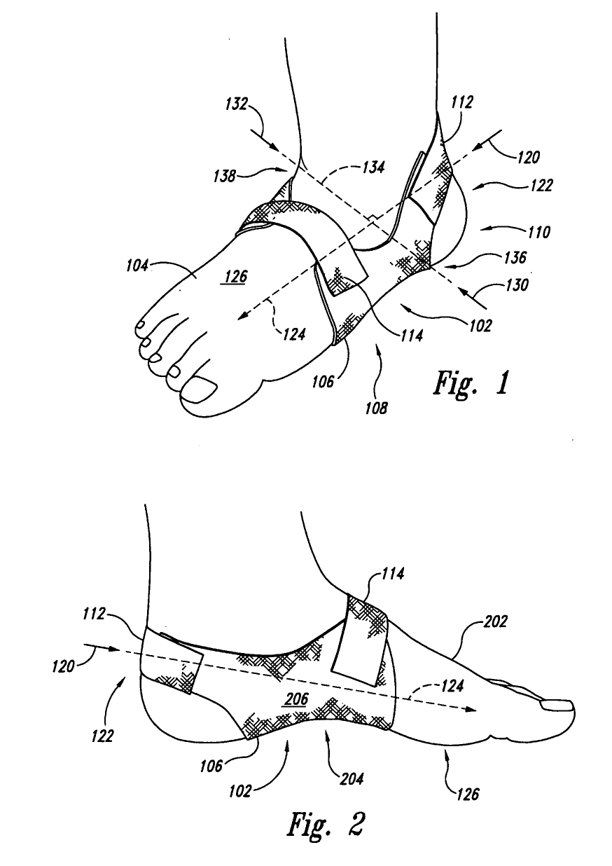

FIG. 2 shows a side view of support 102 applied to a human left foot 202, in accordance with an exemplary embodiment of the present invention. Body 106 of support 102 may be applied to embrace a lower mid-foot portion 204 of foot 202 and to simultaneously apply medial and lateral compression of a heel portion 110 of foot 202 based on the cooperative actions of first fastening member 112, second fastening member 114, and flexible body member 106. FIG. 2 also shows a first side 206, or outer surface, of body member 106 that may include a rubber tread, a layer, or coating that provides a non-skid surface for safety when support 102 is worn within or without a boot, shoe, or sandal. Further, for an individual suffering from bunion pain, first fastening member 112 may be used as an anchoring point for a bunion splint. In one example, a fusiform strap may be inserted between the hallux (big toe) and second digit. The two ends of the fusiform strap may then be passed medially at which point the two ends could be crossed and pulled with a medial-proximal force and attached to first fastening member 112. In one embodiment, the two ends of the fusiform strap may be attached to corresponding locations on first fastening member 112. The anchored bunion splint may include NEOPRENE or other suitable material.

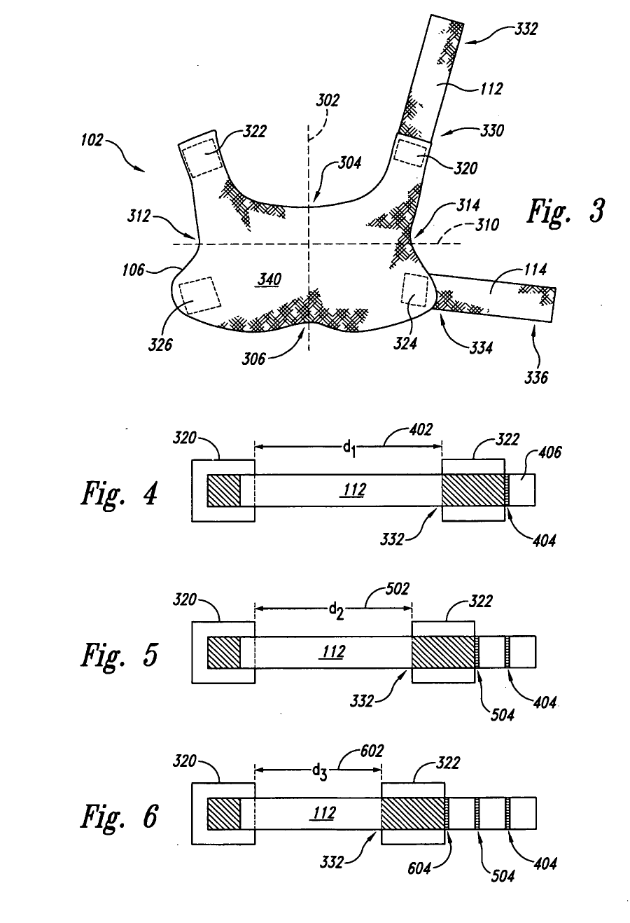

FIG. 3 shows a plan view of support 102, in accordance with an exemplary embodiment of the present invention. Body 106 has symmetry across a central first axis 302, where a body member first end 304 and a body member second end 306 are disposed oppositely on first axis 302. Body 106 also has symmetry along a second axis 310, where a first side 312 and a second side 314 are disposed oppositely across first axis 302 and along second axis 310. First axis 302 and second axis 310 may be perpendicular to each other. Body member first end 304 has a first fastening site 320 and a second fastening site 322 disposed symmetrically across first axis 302. Body member second end 306 has a third fastening site 324 and a fourth fastening site 326 distributed symmetrically across first axis 302. The dimensions of body member 106 are such that the linear distance along the body member from the first end 304 to the second end 306 is shorter than the linear distance along the body member from the first side 312 to the second side 314. Although symmetrical across axis 302, body member 106 second end 306 has a different curvature than first end 304, wherein the curvature of send end 306 defines a notch or cutout region in the direction of first end 304. The notch region provides a reduction in pressure on a widening mid-foot portion 204 of foot 104 in a direction away from first end 304.

First fastening member 112 includes a first end 330 and a second end 332, where the first fastening member first end 330 may be fixedly attached to the body member first fastening site 320 and the first fastening member second end 332 may be selectively attached to the body member second fastening site 322. First fastening member second end 332 may be released from attachment to body member second fastening site 322, and or may be selectively attached to body member second fastening site 322 at various points along first fastening member 112 in order to allow a selective amount of medial and/or lateral compression. Similarly, second fastening member 114 includes a first end 334 and a second end 336, the second fastening member first end 334 being fixedly attached to the body member third fastening site 324 and the second fastening member second end 336 may be selectively attached to the body member fourth fastening site 326. Second fastening member second end 336 may be released from attachment to body member fourth fastening site 326, and or may be selectively attached to body member fourth fastening site 326 at various points along second fastening member 114 in order to allow a selective amount of medial and/or lateral compression.

The amount of medial and lateral compression may be selectively adjusted by changing the attachment point of first fastening member second end 332 to the body member second fastening site 322, and/or by attachment point of second fastening member second end 336 to the body member fourth fastening site 326. First fastening member 112 and second fastening member 114 may each be composed of a medium having a plurality of attachment points along the respective second end (332, 336).

In one embodiment, the fastening members (112, 114) may be constructed out of VELCRO straps and fastening sites (322, 326) may be constructed out of complementary VELCRO patches which permit continuous attachment of the strap and patch along the length of the respective fastening member (112, 114). The VELCRO straps and patches may be swapped in position for either or both first and second fastening members (112, 114). In another embodiment, laces may be threaded through or attached to first and second ends (320, 322) to provide an adjustable tension based on the formation of a knot connecting the laces, for example. A second side 340, or interior surface, of body member 106 may include a layer or coating that is different from the first side 206 of body member 106 as shown in FIG. 2. Second side 340 may form an interior region of support 102 and may include a moisture absorbent material to communicate perspiration away from the skin surface of a user. This removal of perspiration may prolong the life of support 102 while providing added comfort to a user. Body member 106 may include ⅛″ or ¼″ NEOPRENE material, while fastening members (112, 114) and fastening sites (322,326) may include VELCRO fastener or closure materials. The NEOPRENE material comprising the core of body 106 may be double or single sided and include a colored lining on either or both sides.

FIG. 4 shows an exemplary attachment of first attachment member 112 positioned to hold first fastening site 320 and second fastening site 322 on body 306 at a first distance d1 402 to provide a first amount of tension across an operative portion of first attachment member 112 corresponding to a second amount of medial and lateral compression, in accordance with an embodiment of the present invention. First attachment member 112 may include an attachment length indicator 404 to facilitate attachment and/or reattachment of first attachment member 112 second end 332 to body member second fastening site 322. Indicator 404 may be positioned adjacent to a portion of second fastening site 322 to provide consistent attachment and/or reattachment of first attachment member 112 second end 332 to body member second fastening site 322 at a first position corresponding to distance d1 402. An overlapping portion 406 of first attachment member 112 may extend beyond the attachment point to facilitate grasping by the hand of a doctor, user, or prosthetic technician assisting the user.

In a similar manner, FIG. 5 shows an exemplary attachment of first attachment member 112 positioned to hold first fastening site 320 and second fastening site 322 on body 306 at a second distance d2 502 to provide a second amount of tension across an operative portion of first attachment member 112 corresponding to a second amount of medial and lateral compression, in accordance with an embodiment of the present invention. First attachment member 112 may include an attachment length indicator 504 to facilitate attachment and/or reattachment of first attachment member 112 second end 332 to body member second fastening site 322. Indicator 504 may be positioned adjacent to a portion of second fastening site 322 to provide consistent attachment and/or reattachment of first attachment member 112 second end 332 to body member second fastening site 322 at a second position corresponding to distance d2 502.

Finally, FIG. 6 shows an exemplary attachment of first attachment member 112 positioned to hold first fastening site 320 and second fastening site 322 on body 306 at a third distance d3 602 to provide a third amount of tension across an operative portion of first attachment member 112 corresponding to a third amount of medial and lateral compression, in accordance with an embodiment of the present invention. First attachment member 112 may include an attachment length indicator 604 to facilitate attachment and/or reattachment of first attachment member 112 second end 332 to body member second fastening site 322. Indicator 604 may be positioned adjacent to a portion of second fastening site 322 to provide consistent attachment and/or reattachment of first attachment member 112 second end 332 to body member second fastening site 322 at a third position corresponding to distance d3 602.

The three attachment positions described may conform to a prescribed amount of compression determined to be therapeutically beneficial by a medical professional. In one embodiment, first fastening member 112 may include holes where a low-profile post attached to the second attachment site may extend through a portion of fastening member 112 to provide for attachment thereto. Although only three attachment positions are shown, this is not considered limiting since in at least one embodiment the distance between first fastening site 320 and second fastening site 322 on body 306 may be any value continuous from where the fastening sites (320, 322) are touching, to where the fastening sites are disposed on extreme ends of fastening member 112. This variability in attachment positions may accommodate users' feet of varying sizes as well as varying compression requirements. All aspects of the description herein of first attachment member 112 applies to second attachment member 114, where second attachment member 114 may be attached and adjusted separately from or in conjunction with the attachment and/or adjustment of first attachment member 112.

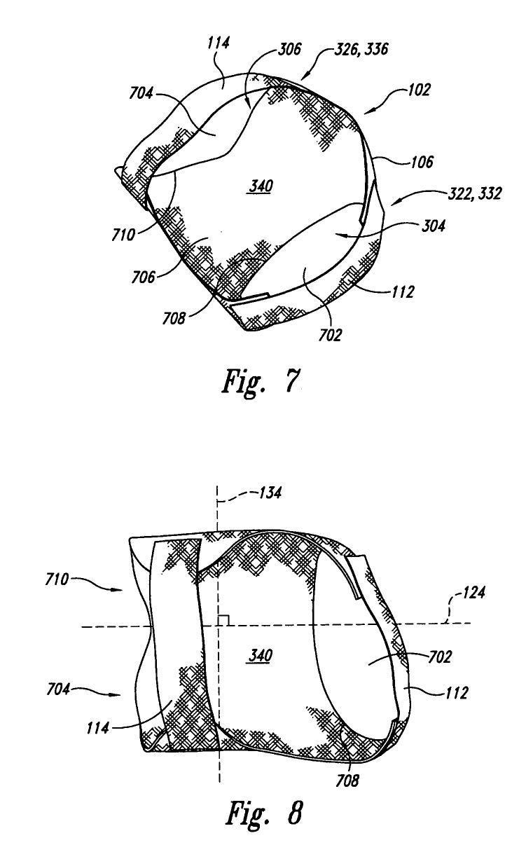

FIG. 7 shows a perspective view of support 102 in a closed position while not embracing a foot, in accordance with an embodiment of the present invention. First attachment member 112 is shown operatively connecting first attachment site 320 and second attachment site 322 to form a first opening or aperture 702 configured to embrace a heel portion of a foot. Second attachment member 114 is shown operatively connecting third attachment site 324 and fourth attachment site 326 to form a second opening or aperture 704 configured to embrace a ball and mid-foot portion 204 of a foot. Finally, the attachment of both first attachment member 112 and second attachment member 114 forms a third aperture 706 configured to embrace a top portion of a foot while allowing a user's leg to extend through aperture 706. In this manner, the first aperture 702 forms a first aperture ring 708 including first fastening member 112, and body member first end 304, where first fastening member 112 second end 332 is attached to second fastening site 322 comprising a first continuous band of material to surround a user's heel. First aperture ring 708 can define an embracing means for embracing a heel portion 110 of a human foot. Aperture ring 708 is disposed on the conical shaped portion of the user's heel so that medial and lateral compression may be applied to the heel. Compression may be felt to a greater degree at the plantarfascia and can serve at least two purposes. The first purpose would be to relieve tension of the plantarfascia, while the second purpose would be to reduce swelling caused by tendonitis, stress fracture, trauma, and/or injury. The second aperture 704 forms a second aperture ring 710 including second fastening member 114, and body member second end 306, where fastening member 114 second end 336 is attached to fourth fastening site 326 comprising a second continuous band of material to surround a user's mid-foot portion 204. In this manner, second aperture ring 710 can define an embracing means for embracing a mid-foot portion of a human foot. Aperture ring 710 is disposed surrounding the narrower ball portion of the user's foot so that medial and lateral compression may be applied to the heel portion 110. By adjusting the attachment position of first fastening member 112 second end 332 with second fastening site 322 as discussed in reference to FIGS. 4-6 the circumference of first aperture ring 708 may be made larger or smaller. Similarly, by adjusting the attachment position of second fastening member 114 second end 336 with second fastening site 326 as discussed in reference to FIGS. 4-6 the circumference of second aperture ring 710 may be made larger or smaller. When first aperture ring 708 or second aperture ring 710 is made larger, less medial, lateral, and/or circumferential compression is applied to the user's heel and mid-foot region, respectively. Conversely, when first aperture ring 708 or second aperture ring 710 is made smaller, more medial, lateral, and/or circumferential compression is applied to the user's heel and mid-foot region, respectively. The resistance to stretching caused by the resilience of body member 106 generates a compressive force between first aperture ring 708 and second aperture ring 710. Body member 106 can be a tensioning means for providing tension between the first aperture ring 708 and the second aperture ring 710. In this manner, the tension between first aperture ring 708 and second aperture ring 710 provides medial and lateral compression of the heel portion of a wearer's foot.

FIG. 8 shows a top view of support 102 in a closed position while not embracing a foot, in accordance with an embodiment of the present invention. First aperture 702, first aperture ring 708, second aperture 704, and second aperture ring 710 are shown along with central axis 124.

In reference to FIGS. 1-8, support 102 may substantially cover the entire arch area of a user's foot, including the medial and lateral aspects of the foot, while exposing the heel and forefoot areas. From a plantar view perspective, support 102 may cover the plantar aspect of the foot between the medial calcaneal tubercle proximally to the metatarso-phalangeal joint parabola distally. From a medial view perspective, support 102 may cover the medial aspect of the foot between the first metatarso-phalangeal joint distally and wrap around the posterior aspect of the heel in a continuous fashion to be anchored on the lateral aspect of the foot. The strap may have a Velcro device configured to attach or anchor to a corresponding Velcro device on the lateral aspect of support 102. From a top view perspective, support 102 may cover the dorsal aspect of the foot. A continuous strap (second attachment member 114) from the medial and the lateral sides of support 102 may anchor on top of the dorsal aspect of the foot.

From a lateral view perspective with the medial strap (first attachment member 112) unsecured, support 102 may cover the lateral aspect of the foot between the fifth metatarso-phalangeal joint distally to the middle of the calcaneus. Support 102 may then have an attachment device attached laterally to receive the medial strap extended around the posterior aspect of the heel from the medial aspect of the foot and anchored laterally. From a lateral view perspective with the medial strap (first attachment member 112) secured, support 102 may cover the lateral aspect of the foot between the fifth metatarso-phalangeal joint distally and show the continuous strap extended from the medial side of the foot continuously over the posterior aspect of the heel and anchored to a corresponding attachment site on the lateral aspect of the foot.

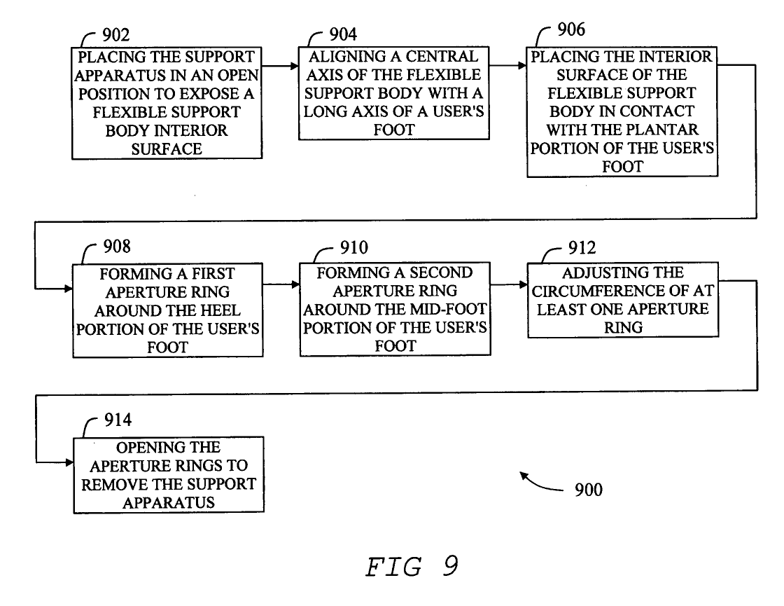

FIG. 9 shows a support apparatus application flow 900 comprising a method of using support 102, in accordance with an embodiment of the present invention. Flow 900 includes placing the heel support in an open position to expose a flexible support body interior surface in operation 902, aligning a central axis of the flexible support body with a long axis of a user's foot in operation 904, and placing the flexible support body interior surface in contact with the plantar or bottom surface portion of the user's foot in operation 906, and forming a first aperture ring around the heel portion of the user's foot in operation 908. Flow 900 continues with forming a second aperture ring around the mid-foot portion of the user's foot, where the formation of the first and second aperture rings providing medial and lateral compression of the user's heel and mid-foot, in operation 910. Flow 900 optionally continues with adjusting the circumference of at least one aperture ring in operation 912. Immediate adjustments can be made. Flow 900 concludes with opening the aperture rings to remove support 102 when the heel and/or arch pain therapy has ended.

In operation 902 through 906 a practitioner or user may lay support 102 open on a flat surface with a side facing upwards which will be in contact with the underside portion of the user's foot. In operation 908 the first fastening member 112, comprising a first strap, is applied to the posterior aspect of the heel as shown in reference to FIGS. 1 and 2. The first strap may be applied so that the medial aspect of support 102 will be positioned by lightly pulling on the strap maintaining tension and brought around from the medial aspect of the foot, posteriorly around the back of the heel and attached to the lateral aspect of the heel to the Velcro strap counterpart. In a similar manner, second fastening member 114, comprising a second strap, will be applied over the top of the foot. The second strap will be closed from the dorso-medial aspect of the foot across the top of the foot and secured on the lateral aspect of the foot. When support 102 is applied correctly, it should begin to provide immediate relief or immediate reduction in pain. The decrease or elimination of pain indicates the proper fit and compression. The device can be worn over a sock or on a bare foot, with or without shoes, boots, or sandals. This also gives the added ability to wear with sandals or shoes without a heel counter.

Support 102, hereinafter the device, can be used while active, at rest, or while sleeping. Examples of use while active include playing sports, prolonged standing, walking, and increased physical activities. The device can be used for both water and non-water sports and/or activities including scuba diving, or water skiing, water polo and other aquatic activities, soccer, basketball, football, volleyball, skiing, baseball, and snowboarding. The device can be worn prior to physical activity to reduce the chance of injury to the heel/arch area and to control chronic arch/heel pain, where the device may be applied at the first sign of heel and/or arch pain for immediate pain control. Accordingly, several advantages are provided including ease of use and application, immediate pain control, stabilization of the foot, physical shock isolation through increased cushioning, and keeping the foot warm. Support 102 may be washable and reusable.

Other objects and advantages may be related to a product display on a retail store shelf. Such a product display may show the product mounted on a mannequin or “plastic” foot model providing a visual aid and instruction on proper application of support 102. Color coding of either the outer or inner lining surfaces may be used to more easily distinguish the support 102 size and facilitate rapid selection by a practitioner, assistant, or consumer. For example, a red surface may correspond to a small size, a blue surface may correspond to a medium size, a green surface may correspond to a large size, and a black or natural NEOPRENE surface may correspond to an extra-large size. Combinations of color and/or alpha-numeric markings may be used to distinguish sizes for men, women, and children, where a smaller men's size may be equivalent to a larger woman's size. Alternatively, combinations of color, lines, and/or other markings or the absence of such indicators may be used to distinguish the type of closure used such as VELCRO, laces, eye-and-button, etc. The ambidextrous capability of application to either left or right feet provides for more universal application, better economy for consumers, and lower stock requirements for retailers.

### Case Study

A case-study was performed using a support 102 according to an embodiment of the present invention. This case-study demonstrated pain reduction benefits with a patient who suffered an avulsion fracture of the fifth metatarsal base (non-Jones' Fracture). A Jones' fracture is a fracture that is perpendicular to the shaft of the 5th metatarsal and typically requires surgical intervention. However, support 102 may be used after removal of a cast or even worn in conjunction with a walking cast for a Jones' fracture patient. This result indicates a favorable result for patients with stress fractures of other metatarsal shafts as well.

Additional advantages for patients with avulsion fracture of the 5th metatarsal base would be the low profile of the support or splint. A patient would be able to wear support 102 in their own shoes as well as in a walking type immobilization cast. Another application for this device would be as an anchoring device for bunion splints. This would reduce the bulkiness in all applications whether used for treating fractures, bunions, or plantarfasciitis.

Embodiments described above illustrate but do not limit the invention. Many other variations are possible without departing from the scope of the disclosed invention. For example, pre-cut strapping may be used as fastening members for limited or single use. Exemplary embodiments show the number of Velcro straps used for anchoring the device to the foot are shown to be two, but may be three or more. The variation on anchoring devices may not be limited to Velcro, laces, fasteners, slip on with nylon mesh, glue, or any adhesives. It should also be understood that numerous modifications and variations are possible without departing from the principles of the present invention. Accordingly, the scope of the invention is defined only by the following claims, which are incorporated into this section by reference.

I claim:

1. A heel support apparatus, comprising:
a flexible body member having symmetry across a central first axis, the body member having a first end and a second end disposed oppositely on the first axis, the body member having a first side and a second side disposed oppositely across the first axis, the body member first end having a first fastening site and a second fastening site disposed symmetrically across the first axis, the body member second end having a third fastening site and a fourth fastening site disposed symmetrically across the first axis;
a first fastening member including a first end and a second end, the first fastening member first end configured to be fixedly attached to the body member first fastening site and the first fastening member second end configured to be selectively attached to the body member second fastening site; and
a second fastening member including a first end and a second end, the second fastening member first end being fixedly attached to the body member third fastening site and the second fastening member second end being selectively attached to the body member fourth fastening site.

2. The apparatus of claim 1, wherein selectively attaching the first fastening member second end to the second fastening site forms a first aperture ring.

3. The apparatus of claim 2, wherein the first aperture ring is configured to embrace a heel portion of a human foot.

4. The apparatus of claim 1, wherein selectively attaching the second fastening member second end to the fourth fastening site forms a second aperture ring.

5. The apparatus of claim 4, wherein the second aperture ring is adapted to embrace a mid-foot portion of a human foot.

6. The apparatus of claim 2,
wherein the first aperture simultaneously provides medial and lateral compression of a heel portion.

7. The apparatus of claim 2,
wherein the body member covers an entire arch area of a human foot.

8. The apparatus of claim 1,
wherein the body member is constructed from NEOPRENE having a thickness of one of one-eighth inch and one-quarter inch.

9. The apparatus of claim 1,
wherein the linear distance along the body member from the first end to the second end is shorter than the linear distance along the body member from the first side to the second side.

10. The apparatus of claim 1,
wherein the first fastening site, the second fastening site, the third fastening site, and the fourth fastening site are disposed on the same side of the body member.

11. The apparatus of claim 1,
wherein the body member first end defines a first curvature with symmetry across the first axis, the body member second end defining a second curvature across the first axis, the first curvature being different from the second curvature, the body member second end curvature defining a notch in the direction of the first end.

12. The apparatus of claim 1,
wherein the first fastening member and the second fastening member include at least one material selected from the group consisting of nylon mesh, laces, and VELCRO.

13. A method of applying a heel support, comprising the operations of:
placing the heel support in an open position to expose a flexible support body interior surface;
aligning a central axis of the flexible support body with a long axis of a foot, the foot having a plantar portion, a heel portion, and a mid-foot portion;
placing the flexible support body interior surface in contact with the plantar portion of the foot;
forming a first aperture ring around the heel portion of the foot; and
forming a second aperture ring around the mid-foot portion of the foot, the formation of the first and second aperture rings providing medial and lateral compression of the heel portion.

14. The method of claim 13, further comprising:
adjusting the circumference of at least one aperture ring.

15. The method of claim 13, further comprising:
opening the aperture rings to provide removal of the support apparatus.

16. A support apparatus for a human foot, comprising:
a first embracing means for embracing a heel portion of a human foot;
a second embracing means for embracing a mid-foot portion of the human foot; and
a tensioning means for providing tension between the first embracing means and the second embracing means, tension between the first embracing means and the second embracing means providing medial and lateral compression of the heel portion.

17. The support apparatus of claim 16,
wherein the first embracing means defining a first aperture ring, and
wherein the second embracing means defining a second aperture ring.

18. The support apparatus of claim 16, wherein the fat pad portion of the foot may be properly repositioned and the plantarfascia portion of the foot may be at least one of supported and relaxed.

19. The support apparatus of claim 16, wherein the tensioning means including a resilient flexible body member.

20. The support apparatus of claim 19, wherein the resilient flexible body member includes NEOPRENE.
